(19) United States
(12) Patent Application Publication
(10) Pub. No.: US 2008/0033398 A1
(43) Pub. Date: Feb. 7, 2008

(54) DEVICE AND METHOD FOR ENHANCING IMMUNE RESPONSE BY ELECTRICAL STIMULATION

(75) Inventors: Steven G. Reed, Bellevue, WA (US); Rhea N. Coler, Seattle, WA (US)

Correspondence Address: SEED INTELLECTUAL PROPERTY LAW GROUP PLLC, 701 FIFTH AVE, SUITE 5400, SEATTLE, WA, 98104, US

(73) Assignee: TRANSCUTANEOUS TECHNOLOGIES INC., Tokyo (JP)

(21) Appl. No.: 11/617,606

(22) Filed: Dec. 28, 2006

Related U.S. Application Data
(60) Provisional application No. 60/755,361, filed on Dec. 29, 2005.

(51) Int. Cl. A61N 1/30; A61N 1/32

(52) U.S. Cl. 604/522; 607/3

(57) ABSTRACT

A device and a method are provided to enhance an immune response to a vaccine or immunogen by electrical stimulation of a biological interface in conjunction with administration of the vaccine or immunogen.

### CROSS-REFERENCE TO RELATED APPLICATIONS

This application claims the benefit under 35 U.S.C. § 119(e) of U.S. Provisional Patent Application No. 60/755,361 filed Dec. 29, 2005, where this provisional application is incorporated herein by reference in its entirety.

### BACKGROUND

1. Field

This disclosure generally relates to enhancing the immune response to a vaccine and immunogen, and more particularly to methods for increasing the potency of a vaccine or immunogen by electrical stimulation at the site of administration of the vaccine or immunogen.

2. Description of the Related Art

The immune system in higher organisms, such as mammals, is specialized to protect the organism from the consequences of exposure to foreign materials or external biological influences. The immune system may thus, for example, protect the body against bacterial or viral infection, or the effect of cancer cells or other foreign pathogenic substances. The immune system provides both an innate immunity and an immunity that can adapt to infections or other challenges to the system. Innate immunity allows the organism to quickly respond to exposure to foreign organisms or materials. The adaptive immune system enables the organism to develop a long-term ability to respond to exposure to specific organisms or foreign materials. These two systems act to protect the body from challenges resulting from exposure to pathogenic organisms and foreign substances.

Vaccines or immunogens are preparations of antigenic materials used to stimulate the immune system to produce immunity to disease within the body. Vaccination or immunization by administration of a vaccine or immunogen is widely used to protect against a variety of infectious diseases. Administration of such materials leads to prevention or amelioration of the effects of infection by naturally occurring infectious organisms.

Upon administration of a vaccine or immunogen, the immune system identifies the agent as a foreign material. Response to the foreign material leads to the production of antibodies and T-cells specifically targeted to the particular foreign material. The response to the foreign material, once developed, typically prevents subsequent infection by that material.

The innate and the adaptive immune systems each respond to the stimulus. In some respects, the two systems also work together. Activation of certain cells within the adaptive immune system, for example, requires a secondary signal, which may be provided by certain cells, termed dendritic cells, within the innate immune system. The secondary signal may also be provided artificially by administration of adjuvants, which are chemicals that stimulate an immune response.

Challenges relating to the effective distribution and use of vaccines and immunogen preparations include the cost of development and the production and availability. When it is production cost or a shortage of vaccine or immunogen that limits broad availability and use of such materials within both the developed and the developing nations of the world, effective methods for increasing the potency of such materials are needed. One approach to effectively decreasing cost and increasing availability is the use of adjuvants to increase the potency of a vaccine or immunogen, thus allowing the administration of lower doses. However, adding such materials to a vaccine or immunogen preparation changes the composition, often leading to lengthy and costly clinical trials and regulatory review. For each vaccine or immunogen preparation, or for each adjuvant composition, such trials and review would be required. There thus remains a need for a simple, cost-effective approach to increasing the potency of a vaccine or immunogen preparation that does not alter the composition of, but is effective in conjunction with, administration of any vaccine or immunogen preparation.

### BRIEF SUMMARY

In at least one embodiment, a device operable to enhance the immune response of a mammal to a vaccine or immunogen comprises a first electrode assembly comprising a first electrode element and a second electrode assembly comprising a second electrode element, the device operable to supply an electrical potential to a biological interface of the mammal. In certain embodiments, the first electrode assembly further comprises a first electrolyte reservoir comprising a first electrolyte composition. In other embodiments, the second electrode assembly further comprises a second electrolyte reservoir comprising a second electrolyte composition. The device may further include a means for affixing the device to the biological interface, such as an adhesive. The device may further be in the form of a patch.

In at least one embodiment, a method for enhancing the immune response of a mammal to a vaccine or immunogen comprising: injecting the vaccine or immunogen through the biological interface of the mammal; affixing the device to the biological interface; and applying an electrical potential. The electrical potential may be applied at a constant level or in pulses. In certain embodiments, more than one vaccine or immunogen may be injected. In other embodiments, an adjuvant may also be injected or otherwise supplied transdermally. The device may be affixed to the biological interface at or near the site of injection of the vaccine or immunogen.

### BRIEF DESCRIPTION OF SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1A is a top front view of an electrode system according to one illustrated embodiment.

FIG. 1B is a top plan view of an electrode system according to one illustrated embodiment.

FIG. 2 is a schematic diagram of an iontophoresis device according to the electrode system of FIGS. 1A and 1B comprising an active electrode assembly and a counter electrode assembly, according to one illustrated embodiment.

FIG. 3 is a schematic diagram of an iontophoresis device of FIG. 2 positioned on a biological interface, with an optional outer release liner removed to expose an active agent, according to another illustrated embodiment.

FIG. 4A is a schematic diagram of a device according to the electrode system of FIGS. 1A and 1B to supply an electrical potential, wherein the device comprises a first electrode assembly and a second electrode assembly, each assembly comprising an electrode and an electrolyte reservoir, according to one illustrated embodiment.

FIG. 4B is a schematic diagram of the device of FIG. 4A further including a housing material, according to another illustrated embodiment.

FIG. 4C is a schematic diagram of the device of FIG. 4B positioned on a biological interface, with the outer release liners removed to expose the electrolyte reservoirs, according to a further illustrated embodiment.

FIGS. 5A-5C show the responses of guinea pigs to injection of tetanus toxoid alone (FIG. 5A); to injection of tetanus toxoid followed by a patch with saline (FIG. 5B); and to injection of tetanus toxoid followed by a patch with MPL adjuvant (FIG. 5C).

FIGS. 6A-6F show responses of guinea pigs to injection of Fluzone vaccine; FIGS. 6A-6C show responses in the absence of electrical stimulation; FIGS. 6D-6F show responses with electrical stimulation.

### DETAILED DESCRIPTION

In the following description, certain specific details are included to provide a thorough understanding of various disclosed embodiments. One skilled in the relevant art, however, will recognize that embodiments may be practiced without one or more of these specific details, or with other methods, components, materials, etc. In other instances, well-known structures associated with controllers, including but not limited to voltage and/or current regulators, have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.”

Reference throughout this specification to “one embodiment” or “an embodiment” or “another embodiment” means that a particular referent feature, structure or characteristic described in connection with the embodiment is included in at least one embodiment. Thus, the appearances of the phrases “in one embodiment,” or “in an embodiment,” or “in another embodiment” in various places throughout this specification are not necessarily all referring to the same embodiment. Furthermore, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise. Thus, for example, reference to a device including “an electrode element” includes a single electrode element, or two or more electrode elements. Further, for example, reference to an electrically powered device including “a power source” includes a single power source, or two or more power sources. It should also be noted that the term “or” is generally employed as including “and/or” unless the content clearly dictates otherwise.

As used herein and in the claims, “active agent” refers to a compound, drug, molecule, preparation, composition, material, or treatment that elicits, or aids in eliciting, a biological response from any host, animal, vertebrate, or invertebrate, including for example fish, mammals, amphibians, reptiles, birds, and humans. Examples of active agents include a vaccine, an immunological agent, an adjuvant, an antigen, a therapeutic agent, a pharmaceutical agent, a pharmaceutical (e.g., a drug, a therapeutic compound, a pharmaceutical salt, and the like), a non-pharmaceutical (e.g., a cosmetic substance, and the like), a diagnostic agent, or a protein or a peptide, such as insulin, a chemotherapy agent, an anti-tumor agent, or a local or general anesthetic or painkiller.

In some embodiments, the term “active agent” further refers to the active agent, as well as its pharmacologically active salts, pharmaceutically acceptable salts, prodrugs, metabolites, analogs, and the like. In some further embodiment, the active agent includes at least one ionic, cationic, ionizable, and/or neutral therapeutic drug and/or pharmaceutical acceptable salts thereof. In yet other embodiments, the active agent may include one or more “cationic active agents” that are positively charged, and/or are capable of forming positive charges in aqueous media. For example, many biologically active agents have functional groups that are readily convertible to a positive ion or can dissociate into a positively charged ion and a counter ion in an aqueous medium. Other active agents may be polarized or polarizable, that is exhibiting a polarity at one portion relative to another portion. For instance, an active agent having an amino group can typically take the form an ammonium salt in solid state and dissociates into a free ammonium ion (NH4⁺) in an aqueous medium of appropriate pH.

The term “active agent” may also refer to electrically neutral agents, molecules, or compounds capable of being delivered via electro-osmotic flow. The electrically neutral agents are typically carried by the flow of, for example, a solvent during electrophoresis. Selection of the suitable active agents is therefore within the knowledge of one skilled in the relevant art.

In some embodiments, one or more active agents may be selected from analgesics, anesthetics, anesthetics vaccines, antibiotics, adjuvants, immunological adjuvants, immunogens, tolerogens, allergens, toll-like receptor agonists, toll-like receptor antagonists, immuno-adjuvants, immuno-modulators, immuno-response agents, immuno-stimulators, specific immuno-stimulators, non-specific immuno-stimulators, and immuno-suppressants, or combinations thereof.

Non-limiting examples of such active agents include lidocaine, articaine, and others of the -caine class; morphine, hydromorphone, fentanyl, oxycodone, hydrocodone, buprenorphine, methadone, and similar opioid agonists; sumatriptan succinate, zolmitriptan, naratriptan HCl, rizatriptan benzoate, almotriptan malate, frovatriptan succinate and other 5-hydroxytryptamine1 receptor subtype agonists; resiquimod, imiquidmod, and similar TLR 7 and 8 agonists and antagonists; domperidone, granisetron hydrochloride, ondansetron and such anti-emetic drugs; zolpidem tartrate and similar sleep inducing agents; L-dopa and other anti-Parkinson's medications; aripiprazole, olanzapine, quetiapine, risperidone, clozapine, and ziprasidone, as well as other neuroleptica; diabetes drugs such as exenatide; as well as peptides and proteins for treatment of obesity and other maladies.

Further non-limiting examples of active agents include ambucaine, amethocaine, isobutyl p-aminobenzoate, amolanone, amoxecaine, amylocaine, aptocaine, azacaine, bencaine, benoxinate, benzocaine, N,N-dimethylalanylbenzocaine, N,N-dimethylglycylbenzocaine, glycylbenzocaine, beta-adrenoceptor antagonists betoxycaine, bumecaine, bupivicaine, levobupivicaine, butacaine, butamben, butanilicaine, butethamine, butoxycaine, metabutoxycaine, carbizocaine, carticaine, centbucridine, cepacaine, cetacaine, chloroprocaine, cocaethylene, cocaine, pseudococaine, cyclomethycaine, dibucaine, dimethisoquin, dimethocaine, diperodon, dyclonine, ecognine, ecogonidine, ethyl aminobenzoate, etidocaine, euprocin, fenalcomine, fomocaine, heptacaine, hexacaine, hexocaine, hexylcaine, ketocaine, leucinocaine, levoxadrol, lignocaine, lotucaine, marcaine, mepivacaine, metacaine, methyl chloride, myrtecaine, naepaine, octacaine, orthocaine, oxethazaine, parenthoxycaine, pentacaine, phenacine, phenol, piperocaine, piridocaine, polidocanol, polycaine, prilocaine, pramoxine, procaine (NOVOCAINE®), hydroxyprocaine, propanocaine, proparacaine, propipocaine, propoxycaine, pyrrocaine, quatacaine, rhinocaine, risocaine, rodocaine, ropivacaine, salicyl alcohol, tetracaine, hydroxytetracaine, tolycaine, trapencaine, tricaine, trimecaine tropacocaine, zolamine, a pharmaceutically acceptable salt thereof, and mixtures thereof.

As used herein and in the claims, “antigen” or “antigenic” or “antigenicity” refers to a protein, polypeptide or carbohydrate, and the like, that is recognized by the body as foreign and that stimulates the immune system to produce an antibody; as used herein and in the claims, “antigenic determinant”, also commonly referred to as “epitope,” refers to a specific area or structure (that is, an “antigenic site”) on the surface of an antigen that can cause an immune response, thus stimulating production of an antibody that can recognize and bind to the antigenic site or to structurally related antigenic sites. As used herein and in the claims, an “antigenic portion” of an antigen is a portion that is capable of reacting with serum obtained from an individual infected with an organism from which the antigen is derived or with the antigen itself.

As used herein and in the claims, a polypeptide comprising an antigenic determinant that is “similar to” an antigenic determinant located on a specified antigen refers to a polypeptide that elicits an immune response comparable to that elicited by the specified antigen.

As used herein and in the claims, the term “immunogen,” “immunogenic,” or “immunogenicity” refers to any agent that elicits an immune response. “Immunogen” or “immunogenic” further refers to a preparation that contains an antigen or a mixture of antigens used to confer to a subject immunity against a disease caused by an organism from which the antigen or mixture of antigens is derived or to which the antigen or mixture of antigens is related. Examples of an immunogen include, but are not limited to, natural or synthetic (including modified) peptides, proteins, carbohydrates, lipids, oligonucleotides (DNA, RNA, etc.), chemicals, or any other agents that are recognized by the body as foreign and that stimulate the immune system to produce antibodies. An immunogen may be derived from recombinant DNA technology. An immunogen may be administered by injection or by use of a device such as an iontophoretic device, but may also be administered orally or as an aerosol. An immunogen may be administered prior to, concurrent with, or following use of a device described herein.

As used herein and in the claims, the term “vaccine” refers to any substance having an antigen or mixture of antigens on its surface that causes activation of the immune system of a higher organism, such as a mammal. There are a variety of different types of vaccines. Some types are prepared from living organisms that have been cultivated to limit or eliminate their virulent properties. Other types are prepared from living organisms that are related to the virulent organisms, but are not themselves virulent. Yet other types are prepared from inactivated, non-virulent organisms or from antigenic materials that have been purified from the virulent organisms. Vaccines and components of vaccines may be natural, synthetic, or derived from recombinant DNA technology. A vaccine may be administered by injection or iontophoretically, but may also be administered orally or as an aerosol. A vaccine may be administered prior to, concurrent with, or following use of a device described herein.

As used herein and in the claims, the term “vaccination” refers to administration of a vaccine to produce immunity against a disease. As used herein and in the claims, “immunization” refers to administration of an immunogen or an antigen preparation to produce immunity against a disease. Diseases of which the incidence has been controlled or eliminated by vaccination or immunization include diphtheria, pertussis, tetanus, smallpox, chickenpox, influenza, cholera, hepatitis A and B, measles, rubella, yellow fever, mumps, polio, and tuberculosis.

A vaccine may be directed to a single disease or to multiple diseases. Vaccines for use in vaccination against single diseases include, but are not limited to, anthrax vaccine; BCG (for tuberculosis); cholera vaccine; haemophilis b conjugate vaccines (diphtheria protein conjugate, or meningococcal protein conjugate, or tetanus toxoid protein conjugate); hepatitis A vaccine; hepatitis B vaccine; influenza virus vaccine; influenza virus vaccine for types A and B; Japanese encephalitis virus vaccine; measles virus vaccine; meningococcal polysaccharide (serogroups A, C, Y and W-135) vaccine; mumps virus vaccine; pneumococcal vaccine; poliovirus vaccine; human papillomavirus vaccine; rabies vaccine; rotavirus vaccine; rubella virus vaccine; smallpox vaccine; tetanus toxoid; typhoid vaccine; varicella virus (chickenpox) vaccine; yellow fever vaccine; and zoster vaccine. Vaccines for use in vaccination against multiple diseases include, but are not limited to, diphtheria and tetanus toxoids; diphtheria and tetanus toxoids with pertussis vaccine (DTP); DTP with hepatitis B and poliovirus vaccine; haemophilis b conjugate vaccine (meningococcal protein conjugate) with hepatitis B vaccine; hepatitis A and hepatitis B vaccine; measles and mumps virus vaccine; measles, mumps, and rubella virus vaccine (MMR); measles, mumps, rubella, and varicella virus vaccine; meningococcal polysaccharide (serogroups A, C, Y and W-135) and diphtheria toxoid vaccine.

As used herein and in the claims, the term “adjuvant,” or any derivation thereof, refers to an agent that modifies the effect of another agent while having few, if any, direct effects if administered by itself. For example, an adjuvant may alter or affect an immune response, or may increase the potency or efficacy of a pharmaceutically active agent. Administration of an adjuvant in conjunction with a vaccine or immunogen may enhance the response of the immune system to the vaccine or immunogen. Further, adjuvants may be administered, for example, to stimulate a response to an antigen, or to a level of an antigen, that may not normally yield an immune response. Substances that are used, or are under investigation, as adjuvants include, without limitation, TLR (toll-like receptor) agonists (for example, TLR-2, TLR-4, TLR-5, TLR-7, and TLR-9 agonists), lipid A and lipid A analogs (for example, monophosphoryl lipid A (MPL), 3-O-deacylated monophosphoryl lipid A, and aminoalkylglucosaminide 4-phosphate derivatives of lipid A), saponin or derivatives thereof (for example, QS-21), RC-529, CpG, MDP, flagellin, imiquimod, resiquimod and dSLIM. An adjuvant may be administered prior to, concurrent with, or following administration of a vaccine or immunogen. An adjuvant may be administered prior to, concurrent with, or following use of a device described herein.

As used herein and in the claims, the term “agonist” refers to a compound that can combine with a receptor (e.g., a Toll-like receptor, an opioid receptor, and the like) to produce a cellular response. An agonist may be a ligand that directly binds to the receptor. Alternatively, an agonist may combine with a receptor indirectly by forming a complex with another molecule that directly binds the receptor, or otherwise resulting in the modification of a compound so that it directly binds to the receptor.

As used herein and in the claims, the term “antagonist” refers to a compound that can combine with a receptor (e.g., a Toll-like receptor, an opioid receptor, and the like) to inhibit a cellular response. An antagonist may be a ligand that directly binds to the receptor. Alternatively, an antagonist may combine with a receptor indirectly by forming a complex with another molecule that directly binds to the receptor, or otherwise results in the modification of a compound so that it directly binds to the receptor.

As used herein and in the claims, the term “allergen” refers to any agent that elicits an allergic response. Some examples of allergens include but are not limited to chemicals and plants, drugs (such as antibiotics, serums), foods (such as milk, wheat, eggs, etc), bacteria, viruses, other parasites, inhalants (dust, pollen, perfume, smoke), and/or physical agents (heat, light, friction, radiation). As used herein, an allergen may be an immunogen.

As used herein and in the claims, the term “polypeptide” encompasses amino acid chains of any length, including full-length proteins, wherein the amino acid residues are linked by covalent peptide bonds.

As used herein and in the claims, a “variant” is a polypeptide that differs from a native antigen only in conservative substitutions and/or modifications, such that antigenic properties of the native antigen are retained. Such variants may generally be identified by modifying a polypeptide sequence and evaluating the antigenic properties of the modified polypeptide. A “conservative substitution” is one in which an amino acid is substituted for another amino acid that has similar properties. In general, the following groups of amino acids represent conservative changes: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his. Variants may also, or alternatively, be modified by, for example, the deletion or addition of amino acids that have minimal influence on the antigenic properties or structural characteristics of the polypeptide.

As used herein and in the claims, a “fusion protein” or “fusion polypeptide” comprises two or more protein/polypeptide sequences joined via a peptide linkage into a single amino acid chain. The sequences may be joined directly, without intervening amino acids, or by way of a linker amino acid sequence.

As used herein and in the claims, the term “opioid” generally refers to any agent that binds to and/or interacts with opioid receptors. Among the opioid classes examples include endogenous opioid peptides, opium alkaloids (e.g., morphine, codeine, and the like), semi-synthetic opioids (e.g., heroin, oxycodone and the like), synthetic opioids (e.g., buprenorphinemeperidine, fentanyl, morphinan, benzomorphan derivatives, and the like), as well as opioids that have structures unrelated to the opium alkaloids (e.g., pethidine, methadone, and the like).

As used herein and in the claims, the term “analgesic” refers to an agent that lessens, alleviates, reduces, relieves, or extinguishes a neural sensation in an area of a subject's body. In some embodiments, the neural sensation relates to pain, in other aspects the neural sensation relates to discomfort, itching, burning, irritation, tingling, “crawling,” tension, temperature fluctuations (such as fever), inflammation, aching, or other neural sensations.

As used herein and in the claims, the term “anesthetic” refers to an agent that produces a reversible loss of sensation in an area of a subject's body. In some embodiments, the anesthetic is considered to be a “local anesthetic” in that it produces a loss of sensation only in one particular area of a subject's body.

As one skilled in the relevant art would recognize, some agents may act as both an analgesic and an anesthetic, depending on the circumstances and other variables including but not limited to dosage, method of delivery, medical condition or treatment, and an individual subject's genetic makeup. Additionally, agents that are typically used for other purposes may possess local anesthetic or membrane stabilizing properties under certain circumstances or under particular conditions.

As used herein and in the claims, the term “effective amount” or “therapeutically effective amount” includes an amount effective at dosages and for periods of time necessary, to achieve the desired result. The effective amount of a composition containing a pharmaceutical agent may vary according to factors such as the disease state, age, gender, and weight of the subject.

As used herein and in the claims, the terms “vehicle,” “carrier,” “pharmaceutical vehicle,” “pharmaceutical carrier,” “pharmaceutically acceptable vehicle,” “pharmaceutically acceptable carrier,” “diagnostic vehicle,” “diagnostic carrier,” “diagnostically acceptable vehicle,” or “diagnostically acceptable carrier” may be used interchangeably, depending on whether the use is pharmaceutical or diagnostic, and refer to pharmaceutically or diagnostically acceptable solid or liquid, diluting or encapsulating, filling or carrying agents, which are usually employed in pharmaceutical or diagnostic industry for making pharmaceutical or diagnostic compositions. Examples of vehicles include any liquid, gel, salve, cream, solvent, diluent, fluid ointment base, vesicle, liposomes, niosomes, ethasomes, transfersomes, virosomes, cyclic oligosaccharides, non ionic surfactant vesicles, phospholipid surfactant vesicles, micelle, and the like, that is suitable for use in contacting a subject.

In some embodiments, a pharmaceutical vehicle may refer to a composition that includes and/or delivers a pharmacologically active agent, but is generally considered to be otherwise pharmacologically inactive. In some other embodiments, the pharmaceutical vehicle may have some therapeutic effect when applied to a site such as a mucous membrane or skin, by providing, for example, protection to the site of application from conditions such as injury, further injury, or exposure to elements. Accordingly, in some embodiments, the pharmaceutical vehicle may be used for protection without a pharmacologically active agent in the formulation.

Examples of vehicles include degradable or non-degradable polymers, hydrogels, organogels, liposomes, nisomes, ethasomes, transfersomes, virosomes, cyclic oligosaccharides, non-ionic surfactant vesicles, phospholipid surfactant vesicles, micelles, microspheres, creams, emulsions, lotions, pastes, gels, ointments, organogel, and the like, as well as any matrix that allows for transport of an agent across the skin or mucous membranes of a subject. In at least one embodiment, the vehicle allows for controlled release formulations of the compositions disclosed herein.

As used herein and in the claims, the term “membrane” means a boundary, layer, barrier or material, which may, or may not, be permeable. Unless specified otherwise, membranes may take the form a solid, liquid, or gel, and may or may not have a distinct lattice, non-cross-linked structure, or cross-linked structure.

As used herein and in the claims, the term “ion selective membrane” means a membrane that is substantially selective to ions, passing certain ions while blocking passage of other ions. An ion selective membrane, for example, may take the form of a charge selective membrane, or may take the form of a semi-permeable membrane.

As used herein and in the claims, the term “charge selective membrane” means a membrane that substantially passes and/or substantially blocks ions based primarily on the polarity or charge carried by the ion. Charge selective membranes are typically referred to as ion exchange membranes, and these terms are used interchangeably herein and in the claims. Charge selective or ion exchange membranes may take the form of a cation exchange membrane, an anion exchange membrane, and/or a bipolar membrane. A cation exchange membrane substantially permits the passage of cations and substantially blocks anions. Examples of commercially available cation exchange membranes include those available under the designators NEOSEPTA, CM-1, CM-2, CMX, CMS, and CMB from Tokuyama Co., Ltd. Conversely, an anion exchange membrane substantially permits the passage of anions and substantially blocks cations. Examples of commercially available anion exchange membranes include those available under the designators NEOSEPTA, AM-1, AM-3, AMX, AHA, ACH, and ACS, also from Tokuyama Co., Ltd.

As used herein and in the claims, the term “bipolar membrane” means a membrane that is selective to two different charges or polarities. Unless specified otherwise, a bipolar membrane may take the form of a unitary membrane structure, a multiple membrane structure, or a laminate. The unitary membrane structure may include a first portion including cation ion exchange materials or groups and a second portion opposed to the first portion, including anion ion exchange materials or groups. The multiple membrane structure (e.g., two film structure) may include a cation exchange membrane laminated or otherwise coupled to an anion exchange membrane. The cation and anion exchange membranes initially start as distinct structures, and may or may not retain their distinctiveness in the structure of the resulting bipolar membrane.

As used herein and in the claims, the term “semi-permeable membrane” means a membrane that is substantially selective based on a size or molecular weight of the ion. Thus, a semi-permeable membrane substantially passes ions of a first molecular weight or size, while substantially blocking passage of ions of a second molecular weight or size, greater than the first molecular weight or size. In some embodiments, a semi-permeable membrane may permit the passage of some molecules at a first rate, and some other molecules at a second rate different from the first. In yet further embodiments, the “semi-permeable membrane” may take the form of a selectively permeable membrane allowing only certain selective molecules to pass through it.

As used herein and in the claims, the term “porous membrane” means a membrane that is not substantially selective with respect to ions at issue. For example, a porous membrane is one that is not substantially selective based on polarity, and not substantially selective based on the molecular weight or size of a subject element or compound.

A used herein and in the claims, the term “reservoir” means any form or mechanism to retain an electrolyte solution, buffer, element, compound, pharmaceutical composition, diagnostic composition, active agent, and the like, in a liquid state, solid state, gaseous state, mixed state and/or transitional state. For example, unless specified otherwise, a reservoir may include one or more cavities formed by a structure, and may include one or more ion exchange membranes, semi-permeable membranes, porous membranes and/or gels if such are capable of at least temporarily retaining an electrolyte solution, buffer, element or compound. Typically, when a reservoir serves to retain a biologically active agent, it does so prior to the discharge of such agent by electromotive force and/or current to or into the biological interface.

As used herein and in the claims, the term “gel matrix” means a type of reservoir, which takes the form of a three dimensional network, a colloidal suspension of a liquid in a solid, a semi-solid, a cross-linked gel, a non-cross-linked gel, a jelly-like state, and the like. In some embodiments, the gel matrix may result from a three dimensional network of entangled macromolecules (e.g., cylindrical micelles). In some embodiments, a gel matrix may include hydrogels, organogels, and the like. Hydrogels refer to three-dimensional networks of, for example, cross-linked hydrophilic polymers in the form of a gel and substantially composed of water. Hydrogels may have a net positive or negative charge, or may be neutral.

As used herein and in the claims, the term “administration” and all derivations thereof refers to the act of applying or introducing a vaccine, an immunogen, an active agent, compound, drug, substance, preparation, composition, or material to a subject.

As used herein and in the claims, “in conjunction with” and any derivations thereof refer to administration of a vaccine, immunogen, active agent, vehicle, carrier, and the like, simultaneously with, prior to, or subsequent to administration of a further vaccine, immunogen, active agent, vehicle, carrier, and the like.

As used herein and in the claims, the term “subject” generally refers to any host, animal, vertebrate, or invertebrate, and includes fish, mammals amphibians, reptiles, birds, and particularly humans.

The headings provided herein are for convenience only and do not interpret the scope or meaning of the embodiments.

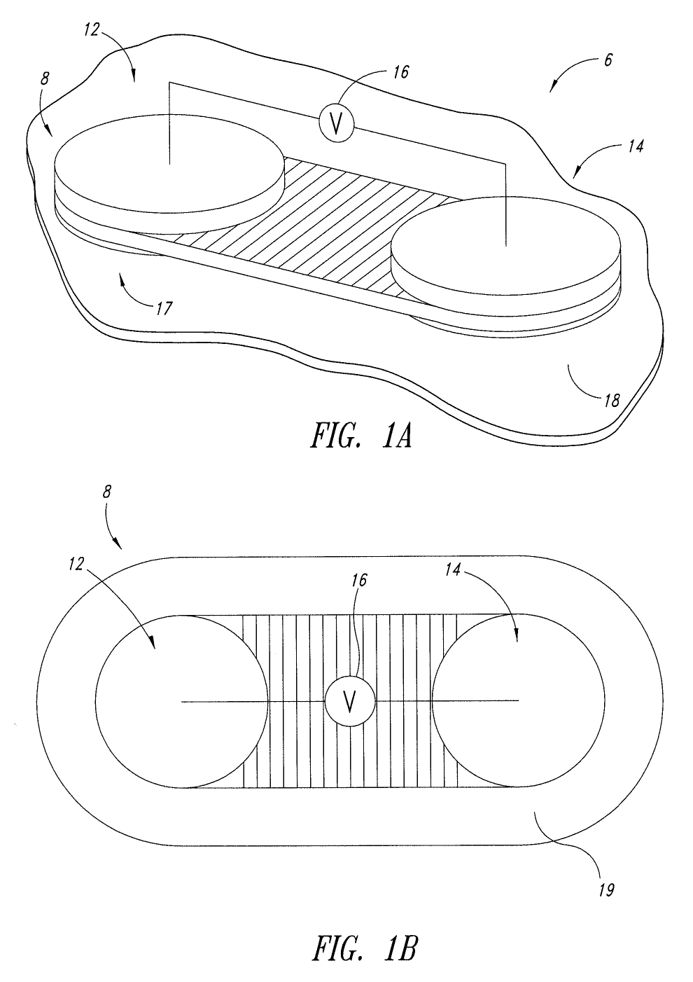

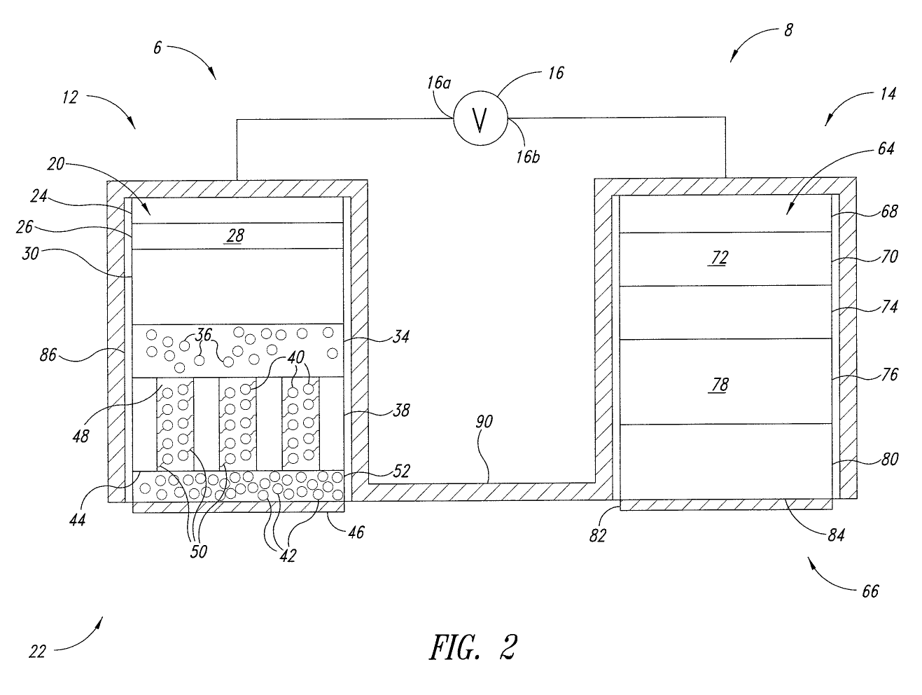

FIGS. 1A and 1B show an exemplary electrode system for application of an electrical stimulus and/or for delivery of one or more active agents. The system 6 includes a device 8 including first (or active) and second (or counter) electrode assemblies 12, 14, respectively, and a power source 16. In certain embodiments, as described herein, the device 8 may be an iontophoretic delivery device. The first (or active) and second (or counter) electrode assemblies 12, 14, are electrically coupleable to the power source 16 to provide and electrical stimulus or to supply, via iontophoresis, an active agent contained in the first (or active) electrode assembly 12 to a biological interface 18, (e.g., a portion of skin or mucous membrane). The device 8 may optionally include a biocompatible backing 19. In some embodiments, the biocompatible backing 19 encases the device 8. In some other embodiments, the biocompatible backing physically couples the device 8 to the biological interface 18 of the subject. In some embodiments, the system 6 is configured to include a physiological salt and to provide an electrical stimulus to the biological interface 18 and the underlying tissue when activated. In other embodiments, the system 6 is configured to transdermally deliver one or more active agents when activated.

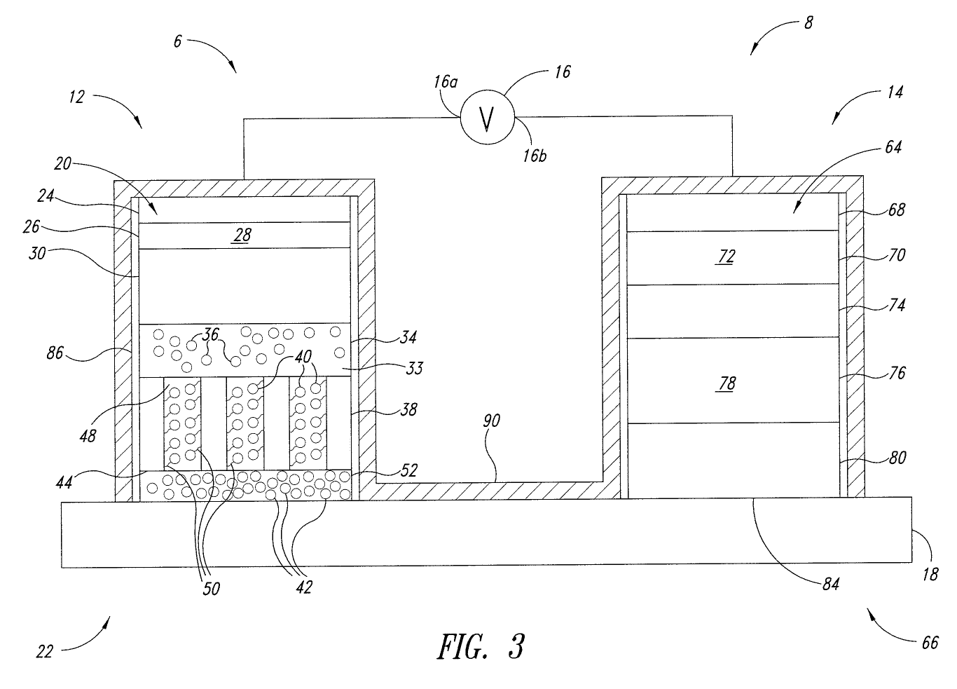

FIGS. 2 and 3 show an exemplary system 6 as an iontophoretic delivery system, wherein device 8 is an iontophoresis device. As shown, an active (or first) electrode assembly 12 may comprise, from an interior 20 to an exterior 22 of the active (or first) electrode assembly 12: an active (or first) electrode element 24, a electrolyte reservoir 26 storing a electrolyte 28, an optional inner ion selective membrane 30, one or more inner active agent reservoirs 34, storing one or more active agents 36, an optional outermost ion selective membrane 38 that optionally caches additional active agents 40, and an optional further active agent 42 carried by an outer surface 44 of the outermost ion selective membrane 38. Each of the above elements or structures will be discussed in detail below.

The active (or first) electrode assembly 12 may comprise an optional inner sealing liner (not shown) between two layers of the active (or first) electrode assembly 12, for example, between the inner ion selective membrane 30 and the inner active agent reservoir 34. The inner sealing liner, if present, would be removed prior to application of the iontophoretic device to the biological surface 18. The active (or first) electrode assembly 12 may further comprise an optional outer release liner 46.

In some embodiments, the one or more active agent reservoirs 34 are loadable with a vehicle and/or composition for transporting, delivering, encapsulating, and/or carrying the one or more active agents 36, 40, 42. In some embodiments, the composition may include a therapeutically effective one or more active agents 36, 40, 42. In some other embodiments, the composition may include an immunogen and/or an adjuvant.

The active (or first) electrode element 24 is electrically coupled to a first pole 16a of the power source 16 and positioned in the active (or first) electrode assembly 12 to apply an electromotive force to transport the active agent 36, 40, 42 via various other components of the active (or first) electrode assembly 12. Under ordinary use conditions, the magnitude of the applied electromotive force is generally that required to deliver the one or more active agents according to a therapeutic effective dosage protocol. In some embodiments, the magnitude is selected such that it meets or exceeds the ordinary use operating electrochemical potential of the iontophoretic delivery device 8. The at least one active electrode element 24 is operable to provide an electromotive force for driving a pharmaceutical composition, or an immunogen or adjuvant, into the subject from the at least one active agent reservoir 34, to the biological interface 18 of the subject.

The active electrode element 24 may take a variety of forms. In one embodiment, the active electrode element 24 may advantageously take the form of a carbon-based active (or first) electrode element. Such may, for example, comprise multiple layers, for example a polymer matrix comprising carbon and a conductive sheet comprising carbon fiber or carbon fiber paper, such as that described in commonly assigned pending Japanese patent application 2004/317317, filed Oct. 29, 2004. The carbon-based electrodes are inert electrodes in that they do not themselves undergo or participate in electrochemical reactions. Thus, an inert electrode distributes current through the oxidation or reduction of a chemical species capable of accepting or donating an electron at the potential applied to the system, (e.g., generating ions by either reduction or oxidation of water). Additional examples of inert electrodes include stainless steel, gold, platinum, capacitive carbon, or graphite.

Alternatively, an active (or first) electrode of sacrificial conductive material, such as a chemical compound or amalgam, may also be used. A sacrificial electrode does not cause electrolysis of water, but would itself be oxidized or reduced. Typically, for an anode a metal/metal salt may be employed. In such case, the metal would oxidize to metal ions, which would then be precipitated as an insoluble salt. An example of such anode includes an Ag/AgCl electrode. The reverse reaction takes place at the cathode in which the metal ion is reduced and the corresponding anion is released from the surface of the electrode.

The electrolyte reservoir 26 may take a variety of forms including any structure capable of retaining electrolyte 28, and in some embodiments may even be the electrolyte 28 itself, for example, where the electrolyte 28 is in a gel, semi-solid or solid form. For example, the electrolyte reservoir 26 may take the form of a pouch or other receptacle, a membrane with pores, cavities, or interstices, particularly where the electrolyte 28 is a liquid. Further, the electrolyte reservoir 26 may be a hydrogel matrix containing ion-exchange functionalities to bind counter-ions, creating a reservoir with properties similar to that of an ion-exchange membrane. One aspect may include derivatives of the hydrogel backbone such as polyvinyl alcohol (PVA) or hydroxyethyl methacrylate (HEMA). Alternative hydrogels may also be used for the hydrogel matrices; for example, derivatives may include carboxylate, cufonate, amine and quaternary amine groups. Derivatives may contain strong and/or weak ionic functionalities. Further, derivatives of the hydrogel backbone may be incorporated with non-derivative backbone hydrogels into the hydrogel matrices.

In one embodiment, the electrolyte 28 comprises ionic or ionizable components in an aqueous medium, which can act to conduct current towards or away from the active electrode element. Suitable electrolytes include, for example, aqueous solutions of salts. Preferably, the electrolyte 28 includes salts of physiological ions, such as, sodium, potassium, chloride, and phosphate.

Once an electrical potential is applied, when an inert electrode element is in use, water is electrolyzed at both the active and counter electrode assemblies. In certain embodiments, such as when the active electrode assembly is an anode, water is oxidized. As a result, oxygen is removed from water while protons (H+) are produced. In one embodiment, the electrolyte 28 may further comprise an anti-oxidant. In some embodiments, the anti-oxidant is selected from anti-oxidants that have a lower potential than that of, for example, water. In such embodiments, the selected anti-oxidant is consumed rather than having the hydrolysis of water occur. In some further embodiments, an oxidized form of the anti-oxidant is used at the cathode and a reduced form of the anti-oxidant is used at the anode. Examples of biologically compatible anti-oxidants include, but are not limited to, ascorbic acid (vitamin C), fumaric acid, lactic acid, malic acid, citric acid, and salts thereof, as well as tocopherol (vitamin E).

As noted above, the electrolyte 28 may take the form of an aqueous solution housed within a reservoir 26, or in the form of a dispersion in a hydrogel or hydrophilic polymer capable of retaining substantial amount of water. For instance, a suitable electrolyte may take the form of a solution of 0.5 M disodium fumarate: 0.5 M polyacrylic acid: 0.15 M anti-oxidant.

The inner ion selective membrane 30 is generally positioned to separate the electrolyte 28 and the inner active agent reservoir 34, if such a membrane is included within the device. The inner ion selective membrane 30 may take the form of a charge selective membrane. For example, when the active agent 36, 40, 42 comprises a cationic active agent, the inner ion selective membrane 30 may take the form of an anion exchange membrane, selective to substantially pass anions and substantially block cations. The inner ion selective membrane 30 may advantageously prevent transfer of undesirable elements or compounds between the electrolyte 28 and the inner active agent reservoir 34. For example, the inner ion selective membrane 30 may prevent or inhibit the transfer of sodium (Na⁺) ions from the electrolyte 28, thereby increasing the transfer rate and/or biological compatibility of the iontophoresis device 8.

The inner active agent reservoir 34 is generally positioned between the inner ion selective membrane 30 and the outermost ion selective membrane 38. The inner active agent reservoir 34 may take a variety of forms including any structure capable of temporarily retaining active agent 36. For example, the inner active agent reservoir 34 may take the form of a pouch or other receptacle, a membrane with pores, cavities, or interstices, particularly where the active agent 36 is a liquid. The inner active agent reservoir 34 further may comprise a gel matrix.

Optionally, an outermost ion selective membrane 38 is positioned generally opposed across the active (or first) electrode assembly 12 from the active (or first) electrode element 24. The outermost membrane 38 may, as in the embodiment illustrated in FIGS. 2 and 3, take the form of an ion exchange membrane having pores 48 (only one called out in FIGS. 2 and 3 for sake of clarity of illustration) of the ion selective membrane 38 including ion exchange material or groups 50 (only three called out in FIGS. 2 and 3 for sake of clarity of illustration). Under the influence of an electromotive force or current, the ion exchange material or groups 50 selectively substantially passes ions of the same polarity as active agent 36, 40, while substantially blocking ions of the opposite polarity. Thus, the outermost ion exchange membrane 38 is charge selective. Where the active agent 36, 40, 42 is a cation (e.g., lidocaine), the outermost ion selective membrane 38 may take the form of a cation exchange membrane, thus allowing the passage of the cationic active agent while blocking the back flux of the anions present in the biological interface, such as skin.

The outermost ion selective membrane 38 may optionally cache active agent 40. Without being limited by theory, the ion exchange groups or material 50 temporarily retains ions of the same polarity as the polarity of the active agent in the absence of electromotive force or current and substantially releases those ions when replaced with substitutive ions of like polarity or charge under the influence of an electromotive force or current.

Alternatively, the outermost ion selective membrane 38 may take the form of semi-permeable or microporous membrane which is selective by size. In some embodiments, such a semi-permeable membrane may advantageously cache active agent 40, for example by employing the removably releasable outer release liner to retain the active agent 40 until the outer release liner is removed prior to use.

The outermost ion selective membrane 38 may be optionally preloaded with the additional active agent 40, such as ionized or ionizable drugs or therapeutic agents and/or polarized or polarizable drugs or therapeutic agents. Where the outermost ion selective membrane 38 is an ion exchange membrane, a substantial amount of active agent 40 may bond to ion exchange groups 50 in the pores, cavities or interstices 48 of the outermost ion selective membrane 38.

The active agent 42 that fails to bond to the ion exchange groups of material 50 may adhere to the outer surface 44 of the outermost ion selective membrane 38 as the further active agent 42. Alternatively, or additionally, the further active agent 42 may be positively deposited on and/or adhered to at least a portion of the outer surface 44 of the outermost ion selective membrane 38, for example, by spraying, flooding, coating, electrostatically, vapor deposition, and/or otherwise. In some embodiments, the further active agent 42 may sufficiently cover the outer surface 44 and/or be of sufficient thickness to form a distinct layer 52. In other embodiments, the further active agent 42 may not be sufficient in volume, thickness, or coverage as to constitute a layer in a conventional sense of such term.

The active agent 42 may be deposited in a variety of highly concentrated forms such as, for example, solid form, nearly saturated solution form, or gel form. If in solid form, a source of hydration may be provided, either integrated into the active (or first) electrode assembly 12, or applied from the exterior thereof just prior to use.

In some embodiments, the active agent 36, additional active agent 40, and/or further active agent 42 may be identical or similar compositions or elements. In other embodiments, the active agent 36, additional active agent 40, and/or further active agent 42 may be different compositions or elements from one another. Thus, a first type of active agent may be stored in the inner active agent reservoir 34, while a second type of active agent may be cached in the outermost ion selective membrane 38. In such an embodiment, either the first type or the second type of active agent may be deposited on the outer surface 44 of the outermost ion selective membrane 38 as the further active agent 42. Alternatively, a mix of the first and the second types of active agent may be deposited on the outer surface 44 of the outermost ion selective membrane 38 as the further active agent 42. As a further alternative, a third type of active agent composition or element may be deposited on the outer surface 44 of the outermost ion selective membrane 38 as the further active agent 42. In another embodiment, a first type of active agent may be stored in the inner active agent reservoir 34 as the active agent 36 and cached in the outermost ion selective membrane 38 as the additional active agent 40, while a second type of active agent may be deposited on the outer surface 44 of the outermost ion selective membrane 38 as the further active agent 42. Typically, in embodiments where one or more different active agents are employed, the active agents 36, 40, 42 will all be of common polarity to prevent the active agents 36, 40, 42 from competing with one another. Other combinations are possible.

The outer release liner may generally be positioned overlying or covering further active agent 42 carried by the outer surface 44 of the outermost ion selective membrane 38. The outer release liner may protect the further active agent 42 and/or outermost ion selective membrane 38 during storage, prior to application of an electromotive force or current. The outer release liner may be a selectively releasable liner made of waterproof material, such as release liners commonly associated with pressure sensitive adhesives.

An interface-coupling medium (not shown) may be employed between the electrode assembly and the biological interface 18. The interface-coupling medium may take, for example, the form of an adhesive and/or gel. The gel may take, for the form of a hydrating gel. Selection of suitable bioadhesive gels is within the knowledge of one skilled in the relevant art.

In the embodiment illustrated in FIGS. 2 and 3, the counter (or second) electrode assembly 14 comprises, from an interior 64 to an exterior 66 of the counter (or second) electrode assembly 14: a counter (or second) electrode element 68, an electrolyte reservoir 70 storing an electrolyte 72, an optional inner ion selective membrane 74, an optional buffer reservoir 76 storing buffer material 78, an optional outermost ion selective membrane 80, and an optional outer release liner 82.

The counter (or second) electrode element 68 is electrically coupled to a second pole 16b of the power source 16, the second pole 16b having an opposite polarity to the first pole 16a. In one embodiment, the counter (or second) electrode element 68 is an inert electrode. For example, the counter (or second) electrode element 68 may take the form of the carbon-based electrode element discussed above.

The electrolyte reservoir 70 may take a variety of forms including any structure capable of retaining electrolyte 72, and in some embodiments may even be the electrolyte 72 itself, for example, where the electrolyte 72 is in a gel, semi-solid or solid form. For example, the electrolyte reservoir 70 may take the form of a pouch or other receptacle, or a membrane with pores, cavities, or interstices, particularly where the electrolyte 72 is a liquid.

The electrolyte 72 is generally positioned between the counter (or second) electrode element 68 and the outermost ion selective membrane 80, proximate the counter (or second) electrode element 68. As described above, the electrolyte 72 may provide ions or donate charges to prevent or inhibit the formation of gas bubbles (e.g., hydrogen or oxygen, depending on the polarity of the electrode) on the counter (or second) electrode element 68 and may prevent or inhibit the formation of acids or bases or neutralize the same, which may enhance efficiency and/or reduce the potential for irritation of the biological interface 18.

The inner ion selective membrane 74 is positioned between and/or to separate the electrolyte 72 from the buffer material 78. The inner ion selective membrane 74 may take the form of a charge selective membrane, such as the illustrated ion exchange membrane that substantially allows passage of ions of a first polarity or charge while substantially blocking passage of ions or charge of a second, opposite polarity. The inner ion selective membrane 74 will typically pass ions of opposite polarity or charge to those passed by the outermost ion selective membrane 80 while substantially blocking ions of like polarity or charge. Alternatively, the inner ion selective membrane 74 may take the form of a semi-permeable or microporous membrane that is selective based on size.

The inner ion selective membrane 74 may prevent transfer of undesirable elements or compounds into the buffer material 78. For example, the inner ion selective membrane 74 may prevent or inhibit the transfer of hydroxy (OH—) or chloride (Cl—) ions from the electrolyte 72 into the buffer material 78.

The optional buffer reservoir 76 is generally disposed between the electrolyte reservoir and the outermost ion selective membrane 80. The buffer reservoir 76 may take a variety of forms capable of temporarily retaining the buffer material 78. For example, the buffer reservoir 76 may take the form of a cavity, a porous membrane, or a gel. The buffer material 78 may supply ions for transfer through the outermost ion selective membrane 80 to the biological interface 18. Consequently, the buffer material 78 may comprise, for example, a salt (e.g., NaCl).

The outermost ion selective membrane 80 of the counter (or second) electrode assembly 14 may take a variety of forms. For example, the outermost ion selective membrane 80 may take the form of a charge selective ion exchange membrane. Typically, the outermost ion selective membrane 80 of the counter (or second) electrode assembly 14 is selective to ions with a charge or polarity opposite to that of the outermost ion selective membrane 38 of the active (or first) electrode assembly 12. The outermost ion selective membrane 80 is therefore an anion exchange membrane, which substantially passes anions and blocks cations, thereby prevents the back flux of the cations from the biological interface. Examples of suitable ion exchange membranes include the previously discussed membranes.

Alternatively, the outermost ion selective membrane 80 may take the form of a semi-permeable membrane that substantially passes and/or blocks ions based on size or molecular weight of the ion.

The outer release liner 82 may generally be positioned overlying or covering an outer surface 84 of the outermost ion selective membrane 80. The outer release liner may protect the outermost ion selective membrane 80 during storage, prior to application of an electromotive force or current. The outer release liner may be a selectively releasable liner made of waterproof material, such as release liners commonly associated with pressure sensitive adhesives. In some embodiments, the outer release liner may be coextensive with the outer release liner 46 of the active (or first) electrode assembly 12.

The iontophoresis device 8 may further comprise an inert molding material 86 adjacent exposed sides of the various other structures forming the active (or first) and counter (or second) electrode assemblies 12, 14. The molding material 86 may advantageously provide environmental protection to the various structures of the active (or first) and counter (or second) electrode assemblies 12, 14. Enveloping the active (or first) and counter (or second) electrode assemblies 12, 14 is a housing material 90.

As best seen in FIG. 3, the active (or first) and counter (or second) electrode assemblies 12, 14 are positioned on the biological interface 18. Positioning on the biological interface may close the circuit, allowing electromotive force to be applied and/or current to flow from one pole 16a of the power source 16 to the other pole 16b, via the active electrode assembly, biological interface 18 and counter (or second) electrode assembly 14.

In use, the outermost active (or first) electrode ion selective membrane 38 may be placed directly in contact with the biological interface 18. Alternatively, an interface-coupling medium (not shown) may be employed between the outermost active (or first) electrode ion selective membrane 38 and the biological interface 18. The interface-coupling medium may take, for example, the form of an adhesive and/or gel. The gel may take, for example, the form of a hydrating gel or a hydrogel. If used, the interface-coupling medium should be permeable by the active agent 36, 40, 42.

In some embodiments, the power source 16 is selected to provide sufficient voltage, current, and/or duration to ensure delivery of the one or more active agents 36, 40, 42 from the reservoir 34 and across a biological interface (e.g., a membrane) to impart the desired physiological effect. The power source 16 may take the form of one or more chemical battery cells, super- or ultra-capacitors, fuel cells, secondary cells, thin film secondary cells, button cells, lithium ion cells, zinc air cells, nickel metal hydride cells, and the like. The power source 16 may, for example, provide a voltage of 12.8 V DC, with tolerance of 0.8 V DC, and a current of 0.3 mA. The power source 16 may be selectively, electrically coupled to the active (or first) and counter (or second) electrode assemblies 12, 14 via a control circuit, for example, via carbon fiber ribbons. The iontophoresis device 8 may include discrete and/or integrated circuit elements to control the voltage, current, and/or power delivered to the electrode assemblies 12, 14. For example, the iontophoresis device 8 may include a diode to provide a constant current to the electrode elements 24, 68.

As suggested above, the one or more active agents 36, 40, 42 may take the form of one or more ionic, cationic, ionizable, and/or neutral drugs or other therapeutic agents. Consequently, the poles or terminals of the power source 16 and the selectivity of the outermost ion selective membranes 38, 80 and inner ion selective membranes 30, 74 are selected accordingly.

During iontophoresis, the electromotive force across the electrode assemblies, as described, leads to a migration of charged active agent molecules, as well as ions and other charged components, through the biological interface into the biological tissue. This migration may lead to an accumulation of active agents, ions, and/or other charged components within the biological tissue beyond the interface. During iontophoresis, in addition to the migration of charged molecules in response to repulsive forces, there is also an electroosmotic flow of solvent (e.g., water) through the electrodes and the biological interface into the tissue. In certain embodiments, the electroosmotic solvent flow enhances migration of both charged and uncharged molecules. Enhanced migration via electroosmotic solvent flow may occur particularly with increasing size of the molecule.

In certain embodiments, the active agent may be a higher molecular weight molecule. In certain aspects, the molecule may be a polar polyelectrolyte. In certain other aspects, the molecule may be lipophilic. In certain embodiments, such molecules may be charged, may have a low net charge, or may be uncharged under the conditions within the active electrode. In certain aspects, such active agents may migrate poorly under the iontophoretic repulsive forces, in contrast to the migration of small more highly charged active agents under the influence of these forces. These higher molecular weight active agents may thus be carried through the biological interface into the underlying tissues primarily via electroosmotic solvent flow. In certain embodiments, the high molecular weight polyelectrolytic active agents may be proteins, polypeptides, or nucleic acids. In other embodiments, the active agent may be mixed with another agent to form a complex capable of being transported across the biological interface via one of the motive methods described above.

In some embodiments, the iontophoretic delivery system 6 includes an iontophoretic delivery device 8 for providing transdermal delivery of one or more therapeutic active agents 36, 40, 42 to a biological interface 18. The delivery device 8 includes active (or first) electrode assembly 12 including at least one active agent reservoir and at least one active electrode element operable to provide an electromotive force to drive an active agent from the at least one active agent reservoir. The delivery device 8 may include a counter (or second) electrode assembly 14 including at least one counter electrode element 68, and a power source 16 electrically coupled to the at least one active and the at least one counter electrode elements 20, 68. In some embodiments, the iontophoretic drug delivery 8 may further include one or more active agents 36, 40, 42 loaded in the at least one active agent reservoir 34.

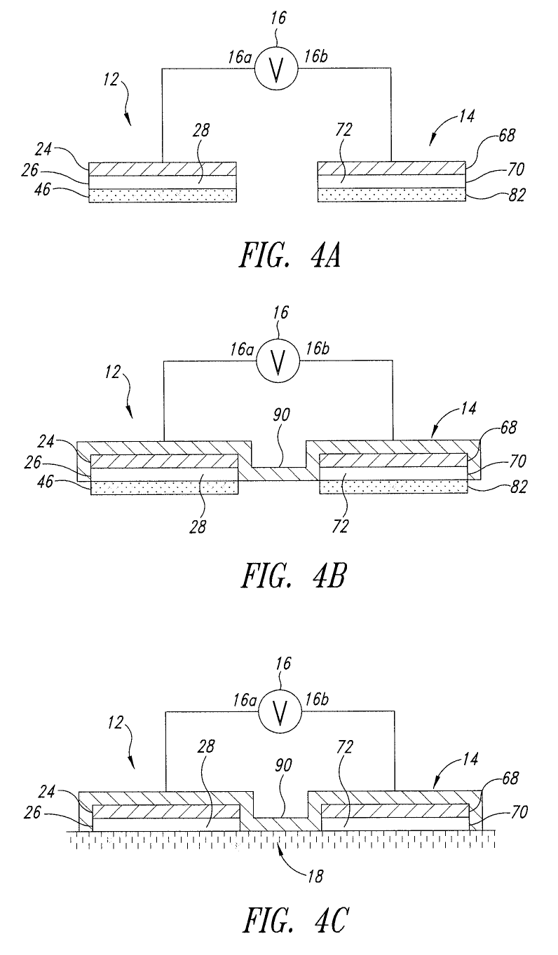

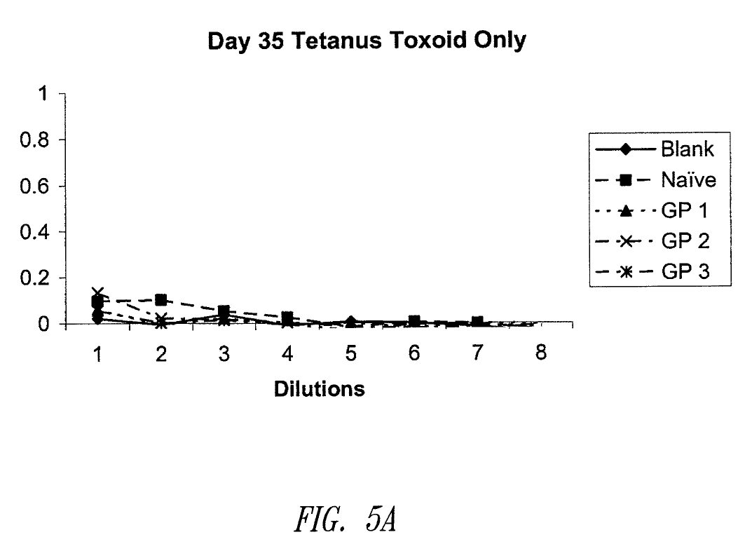

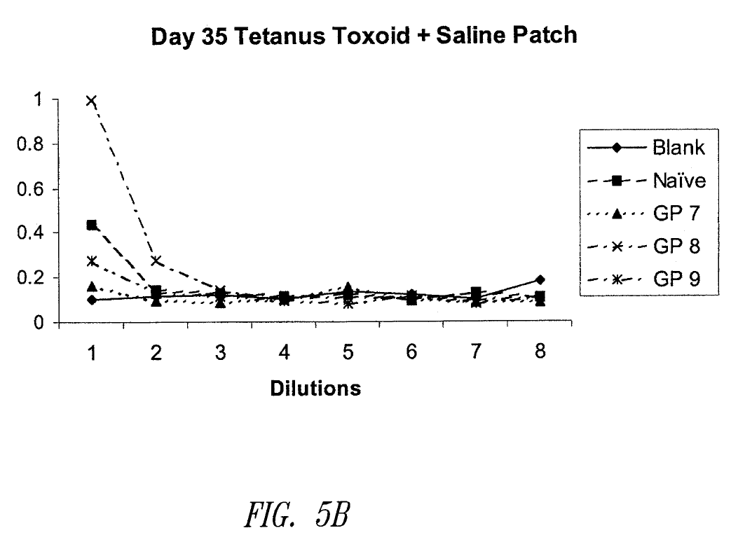

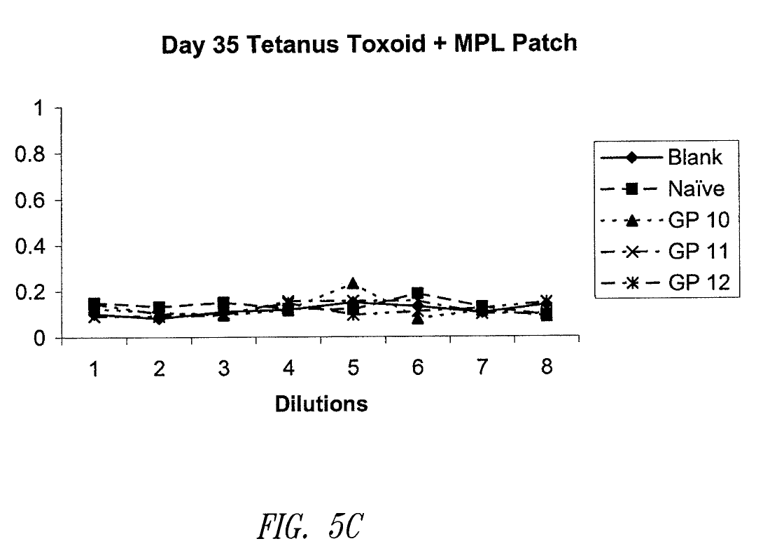

FIGS. 4A-4C show a device comprising first and second electrode assemblies 12, 14, respectively, electrically coupled to a power source 16, operable to provide an electrical stimulus to a biological interface 18 (FIG. 4), such as a portion of a skin or mucous membrane, according to one illustrated embodiment.

In the illustrated embodiments in FIGS. 4A-4C, the first electrode assembly 12 comprises a first electrode element 24, a first electrolyte reservoir 26 storing a first electrolyte 28, and a first release liner 46. Each of the above elements or structures will be discussed below.

The first electrode element 24 is electrically coupled to a first pole 16a of the power source 16 and positioned in the first electrode assembly 12 to apply an electromotive force or current via the components of the first electrode assembly 12 to the biological interface 18. In one embodiment, the first pole 16a is positive.

As discussed above, the first electrode element 24 may take a variety of forms. In one embodiment, the first electrode element 24 may advantageously take the form of a carbon-based first electrode element. Such may, for example, comprise multiple layers, for example a polymer matrix comprising carbon and a conductive sheet comprising carbon fiber or carbon fiber paper, such as that described in commonly assigned pending Japanese application 2004/317317, filed Oct. 29, 2004. Alternatively, a first electrode of sacrificial material may be used. Typically, for an anode a metal/metal salt may be employed. An example of such an anode includes an Ag/AgCl electrode.

Further as discussed above, the first electrolyte reservoir 26 may take a variety of forms including any structure capable of retaining first electrolyte 28, and in some embodiments may even be the electrolyte composition 28 itself, for example where the electrolyte composition 28 is in a gel, semi-solid or solid form. For example, the first electrolyte reservoir 26 may take the form of a pouch or other receptacle, a membrane with pores, cavities or interstices, particularly where the electrolyte composition 28 is a liquid.

In certain embodiments, the electrolyte composition 28 comprises ionic or ionizable components in an aqueous medium which can act to conduct current towards or away from the first electrode element. Suitable electrolytes include aqueous solutions of salts. The electrolyte composition 28 may include, for example, salts of physiological ions, such as sodium, potassium, chloride, and phosphate. In one embodiment, the first electrolyte composition 28 supplies ions for transfer to the biological interface 18. Consequently, the first electrolyte composition may advantageously comprise a salt such as sodium chloride. In some embodiments, the electrolyte composition may comprise further biologically compatible compounds, for example, ascorbic acid, fumaric acid, lactic acid, malic acid, and citric acid, or salts thereof. A suitable electrolyte composition 28 may, for example, take the form of a solution prepared by mixing 0.5M disodium fumarate and 0.5M polyacrylic acid at a ratio of 5:1.

In certain embodiments, the electrolyte composition 28 may be in the form of an aqueous solution. Such aqueous solution housed within the electrolyte reservoir 26 may be in the form of a dispersion in a hydrogel or hydrophilic polymer capable of retaining substantial amounts of water.

The electrolyte composition 28 may provide ions and/or donate charges to prevent or inhibit the formation of gas bubbles (e.g., hydrogen) on the first electrode element 24 in order to enhance efficiency. This elimination or reduction in electrolysis may in turn inhibit or reduce the formation of acids and/or bases (e.g., H⁺ ions, OH⁻ ions) that would otherwise present possible disadvantages such as reduced efficiency and/or possible irritation of the biological interface 18. A suitable electrolyte composition 28 may include a biologically compatible anti-oxidant, such as ascorbate (vitamin C), tocopherol (vitamin E), or sodium citrate, for example, at a concentration of 0.15M.

The first release liner 46 may generally be positioned overlying or covering the first electrolyte reservoir. The liner may protect the first electrolyte composition 28 or the surface of the first electrolyte reservoir 26 during storage, prior to affixing to the biological interface and application of an electromotive force or current. The first release liner 46 may be a selectively releasable liner made of waterproof material, such as release liners commonly associated with pressure sensitive adhesives. Note that the release liner 46 is shown in place in FIGS. 4A and 4B and removed in FIG. 4C.

In the illustrated embodiments in FIGS. 4A-4C, the second electrode assembly 14 comprises a second electrode element 68, a second electrolyte reservoir 70 storing a second electrolyte 72, and a second release liner 82. Each of the above elements or structures will be discussed below.

The second electrode element 68 is electrically coupled to a second pole 16b of the power source 16, the second pole 16b having an opposite polarity to the first pole 16a. In one embodiment, the second pole 16b is negative. The second electrode element 68 may take a variety of forms. In one embodiment, the second electrode element 68 is an inert electrode. For example, the second electrode element 68 may be the carbon-based electrode element discussed above.

The second electrolyte reservoir 70 may take a variety of forms, including any structure capable of retaining second electrolyte 72, and in some embodiments may even be the second electrolyte 72 itself, for example where the second electrolyte 72 is in a gel, semi-solid or solid form. For example, the second electrolyte reservoir 70 may take the form of a pouch or other receptacle, or a membrane with pores, cavities or interstices, particularly where the second electrolyte 72 is a liquid.

The second electrolyte composition 72 is generally positioned between the second electrode element 68 and the surface of the second electrode assembly 14 that is to be affixed to or proximate to the biological interface of the subject. In certain embodiments, the electrolyte composition 72 comprises ionic or ionizable components in an aqueous medium which can act to conduct current toward or away from the second electrode element. Suitable electrolytes include aqueous solutions of salts, as described above, including, for example, salts of physiological ions, such as sodium, potassium, chloride, and phosphate. In one embodiment, the second electrolyte composition 72 supplies ions for transfer to the biological interface 18. Consequently, the second electrolyte composition may advantageously comprise a salt such as sodium chloride. In some embodiments, the electrolyte composition may comprise further biologically compatible compounds, for example, ascorbic acid, fumaric acid, lactic acid, malic acid, and citric acid, or salts thereof. A suitable electrolyte composition 72 may take the form of a solution prepared by mixing 0.155M polyacrylic acid and 0.155M ascorbic acid at a ratio of 3:1.

In certain embodiments, the electrolyte composition 72 may be in the form of an aqueous solution. Such aqueous solution housed within the electrolyte reservoir 70 may be in the form of a dispersion in a hydrogel or hydrophilic polymer capable of retaining substantial amounts of water.

As described above, the second electrolyte composition 72 may provide ions or donate charges to prevent or inhibit the formation of gas bubbles (e.g., hydrogen) on the second electrode element 68 and may prevent or inhibit the formation of acids or bases or neutralize the same, which may enhance efficiency and/or reduce the potential for irritation of the biological interface 18. A suitable electrolyte composition 72 may include a biologically compatible anti-oxidant, such as ascorbate (vitamin C), tocopherol (vitamin E), or sodium citrate, for example, at a concentration of 0.15M.

The second release liner 82 may generally be positioned overlying or covering the front or outer surface of the second electrolyte reservoir 70 or second electrolyte composition 72. The second release liner 82 may protect the front or outer surface of the second electrolyte reservoir 70 or second electrolyte composition 72 during storage, prior to affixing it to the biological interface and application of an electromotive force. The second release liner 82 may be a selectively releasable liner made of waterproof material, such as release liner commonly associated with pressure sensitive adhesives. Note that the second release liner is shown in place in FIGS. 4A and 4B and removed in FIG. 4C. In some embodiments, the second release liner 82 may be coextensive with the first release liner 46 of the first electrode assembly 12.

An interface coupling medium (not shown) may be employed between all or a portion of the surface where electrode assemblies 12 and 14 and housing material 90 in FIG. 4C contact the biological interface 18. The interface coupling medium may, for example, take the form of an adhesive and/or a gel. The gel may, for example, take the form of a hydrating gel.

As discussed above, the power source 16 may take the form of one or more chemical battery cells, super- or ultra-capacitors, fuel cells, secondary cells, thin film secondary cells, button cells, lithium ion cells, zinc air cells nickel metal hydride cells, and the like. The power source 16 may, for example, provide a voltage of 12.8V DC, with tolerance of 0.8V DC, and a current of 0.3 mA. The power source 16 may be selectively electrically coupled to the first and second electrode assemblies 12, 14 via a control circuit (not shown), for example, via carbon fiber ribbons. The device 8 may include discrete and/or integrated circuit elements to control the voltage, current and/or power delivered to the electrode assemblies 12, 14. For example, the device 8 may include a diode to provide a constant current to the electrode elements 24, 68. In certain embodiments, the power source 16 may provide constant voltage, constant current, or constant power. In certain other embodiments, the power source 16 may provide pulses of voltage, pulses of current, or pulses of power.

The above description of illustrated embodiments, including what is described in the Abstract, is not intended to be exhaustive or to limit the claims to the precise forms disclosed. Although specific embodiments of and examples are described herein for illustrative purposes, various equivalent modifications can be made without departing from the spirit and scope of the invention, as will be recognized by those skilled in the relevant art. The teachings provided herein of the invention can be applied to other agent delivery systems and devices, not necessarily the exemplary devices generally described above. Some embodiments may have fewer elements. For example, in certain embodiments, the first electrolyte reservoir 26 and/or the second electrolyte reservoir 70 may be absent. In certain other embodiments, the device may not include first release liner 46 and/or second release liner 82. Other embodiments may include additional structure or elements. For example, some embodiments may include a control circuit or subsystem to control a voltage, current or power applied to the first and second electrode elements 24, 68. Also for example, some embodiments may include an interface layer interposed between the device and the biological interface 18, for example, between the first and/or second electrolyte reservoirs 26, 70 and the biological interface 18. Some embodiments may comprise ion selective membranes, ion exchange membranes, semi-permeable membranes and/or porous membranes, as well as additional reservoirs for electrolytes and/or buffers.

Various electrically conductive hydrogels have been known and used in the medical field to provide an electrical interface to the skin of a subject or within a device to couple electrical stimulus into the subject. Hydrogels hydrate the skin, thus protecting against burning due to electrical stimulation through the hydrogel, while swelling the skin and allowing more efficient transfer of an active component. Examples of such hydrogels are disclosed in U.S. Pat. Nos. 6,803,420; 6,576,712; 6,908,681; 6,596,401; 6,329,488; 6,197,324; 5,290,585; 6,797,276; 5,800,685; 5,660,178; 5,573,668; 5,536,768; 5,489,624; 5,362,420; 5,338,490; and 5,240,995, herein incorporated in their entirety by reference. Further examples of such hydrogels are disclosed in U.S. Patent applications 2004/166147; 2004/105834; and 2004/247655, herein incorporated in their entirety by reference. Product brand names of various hydrogels and hydrogel sheets include Corplex™ by Corium, Tegagel™ by 3M, PuraMatrix™ by BD; Vigilon™ by Bard; ClearSite™ by Conmed Corporation; FlexiGel™ by Smith & Nephew; Derma-Gel™ by Medline; Nu-Gel™ by Johnson & Johnson; and Curagel™ by Kendall, or acrylhydrogel films available from Sun Contact Lens Co., Ltd.

The various embodiments discussed above may advantageously employ various microstructures, for example microneedles. Microneedles and microneedle arrays, their manufacture, and use have been described. Microneedles, either individually or in arrays, may be hollow; solid and permeable; solid and semi-permeable; or solid and non-permeable. Solid, non-permeable microneedles may further comprise grooves along their outer surfaces. Microneedle arrays, comprising a plurality of microneedles, may be arranged in a variety of configurations, for example rectangular or circular. Microneedles and microneedle arrays may be manufactured from a variety of materials, including silicon; silicon dioxide; molded plastic materials, including biodegradable or non-biodegradable polymers; ceramics; and metals. Microneedles, either individually or in arrays, may be used to dispense or sample fluids through the hollow apertures, through the solid permeable or semi-permeable materials, or via the external grooves. Microneedle devices are used, for example, to deliver a variety of compounds and compositions to the living body via a biological interface, such as skin or mucous membrane. In certain embodiments, the active agent compounds and compositions may be delivered into or through the biological interface. For example, in delivering compounds or compositions via the skin, the length of the microneedle(s), either individually or in arrays, and/or the depth of insertion may be used to control whether administration of a compound or composition is only into the epidermis, through the epidermis to the dermis, or subcutaneous. In certain embodiments, microneedle devices may be useful for delivery of high-molecular weight active agents, such as those comprising proteins, peptides and/or nucleic acids, and corresponding compositions thereof. In certain embodiments, for example wherein the fluid is an ionic solution, microneedle(s) or microneedle array(s) can provide electrical continuity between a power source and the tip of the microneedle(s). Microneedle(s) or microneedle array(s) may be used advantageously to deliver or sample compounds or compositions by iontophoretic methods, as disclosed herein. In certain embodiments, for example, a plurality of microneedles in an array may advantageously be formed on an outermost biological interface-contacting surface of an iontophoresis device. Compounds or compositions delivered or sampled by such a device may comprise, for example, high-molecular weight active agents, such as proteins, peptides and/or nucleic acids.

The present invention may be further illustrated by reference to the following examples. These examples are provided by way of illustration only and not by way of limitation of the invention.

### EXAMPLES

### Example 1

### Tetanus Toxoid

Guinea pigs were divided into five groups of three animals each. Each group was immunized with one of the following combinations:

a) 300 μg tetanus toxoid injected subcutaneously (FIG. 4A);

b) 300 μg tetanus toxoid+20 μg MPL adjuvant, formulated together in a volume of 1 mL and injected subcutaneously (not shown);

c) 300 μg tetanus toxoid injected subcutaneously, followed by affixing an iontophoretic patch with saline at the site of injection, delivering a current of 1 milliAmp (density 0.3 mA/cm²) for 30 minutes (average recorded voltage of ˜4 volts) (FIG. 4B);

d) 300 μg tetanus toxoid injected subcutaneously, following by affixing an iontophoretic patch with MPL adjuvant at the site of injection, delivering a current of 1 milliAmp (density 0.3 mA/cm²) for 30 minutes (average recorded voltage of ˜4 volts) (FIG. 4C); and

e) saline injected subcutaneously (not shown).

Blood samples were collected via cardiac puncture using standard protocols. Relative immune responses for certain groups of animals are shown in FIGS. 4A-4C, as identified above. In the iontophoretic patches the first electrolyte composition was formulated with a 5:1 ratio of 0.5M disodium fumarate to 0.5M polyacrylic acid; the second electrolyte composition was formulated with a 3:1 ratio of 0.155M polyacrylic acid to 0.155M ascorbic acid.

The tetanus toxoid absorbed USP and the tetanus toxoid booster were purchased from Sanofi Pasteur. The MPL adjuvant was obtained from Corixa Corporation and was suspended at 1 mg/mL in a solution of 1M HCl and 0.5% sterile triethanolamine formulated at a ratio of 1:50.

### Example 2

### Fluzone

A study was carried out to determine the development of antibody titers to Fluzone influenza vaccine in guinea pigs without or with the use of electrical stimulation. The Fluzone was the 2004-2005 formulation obtained commercially from Aventis Pasteur Inc. The formulation included three strains of influenza: Influenza A/New Caledonia/20/99 (H1N1); Influenza A/Wyoming/03/2003 (H3N2); and Influenza B/Jiangsu/10/2003.

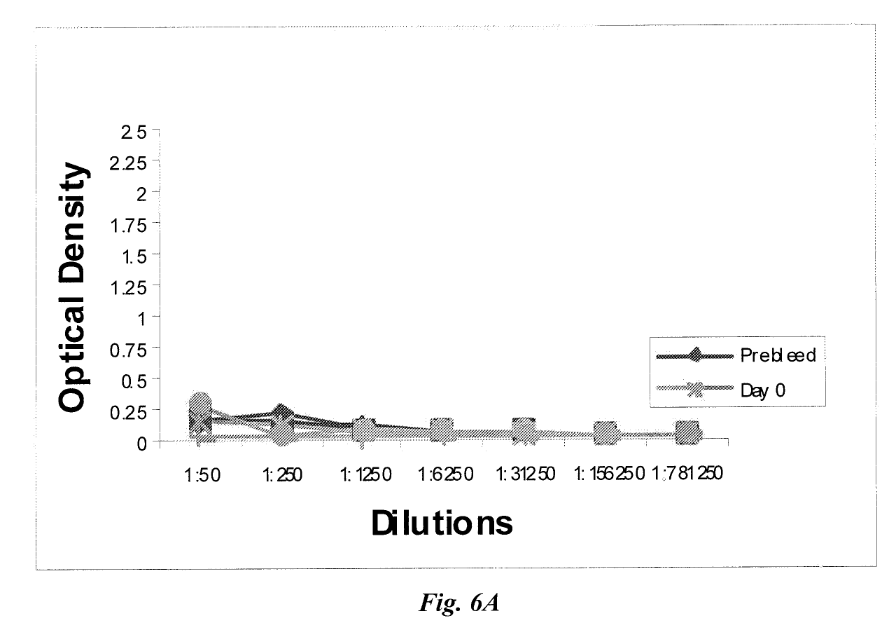

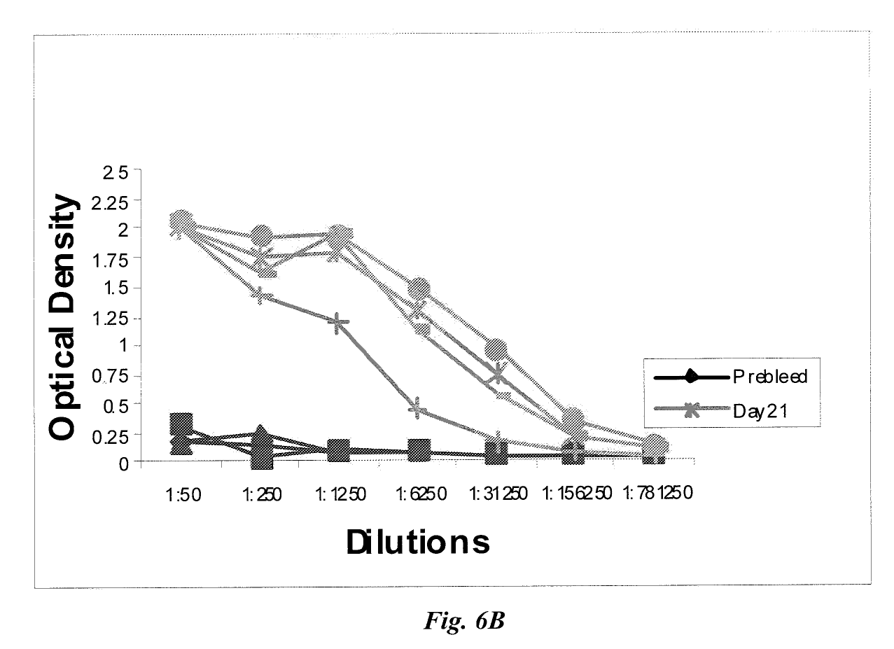

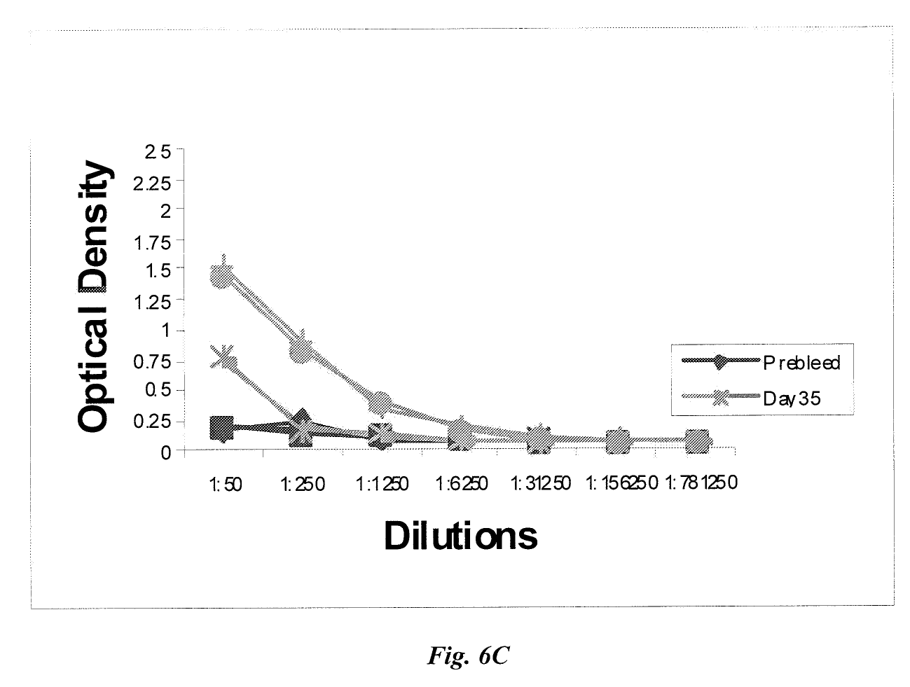

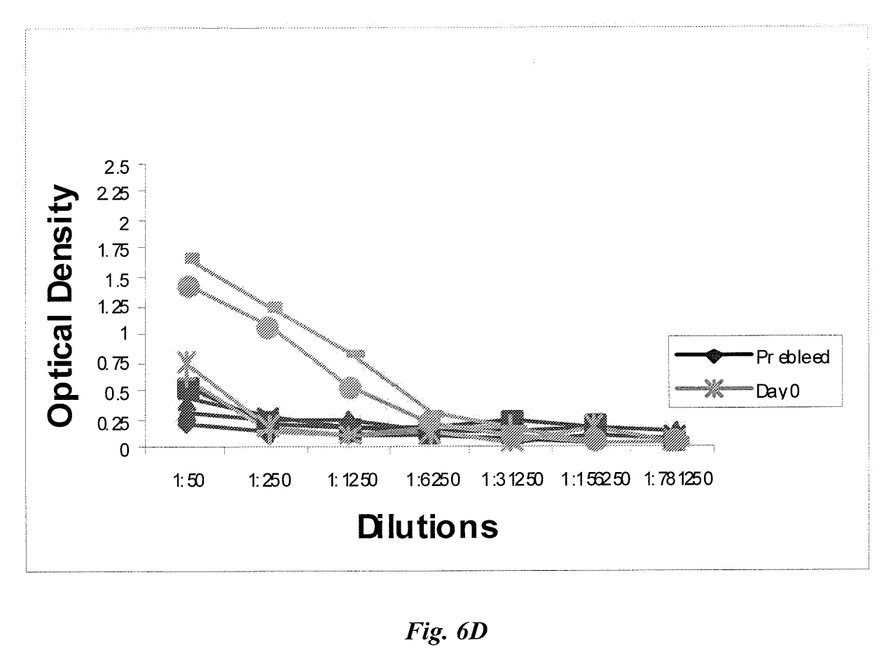

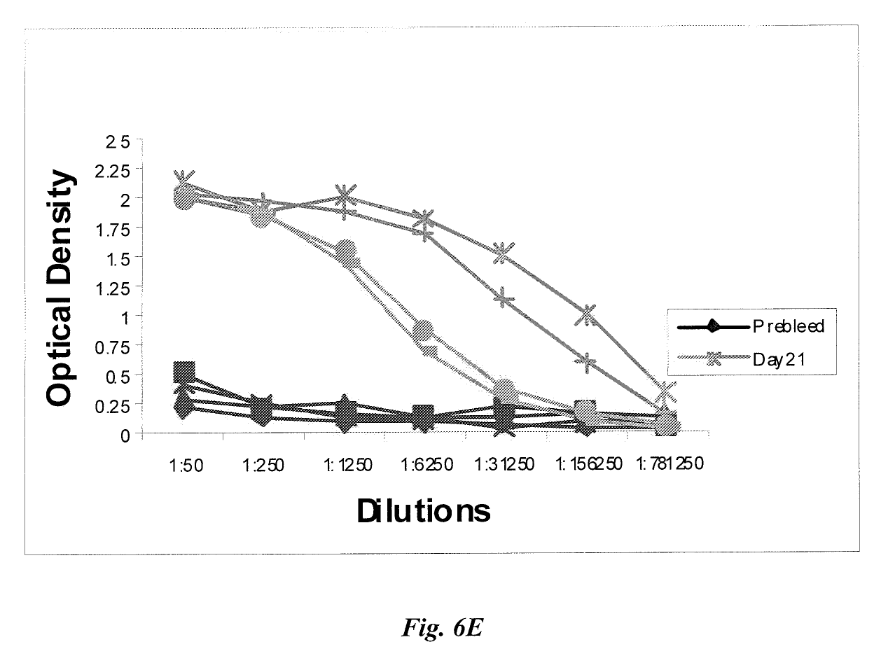

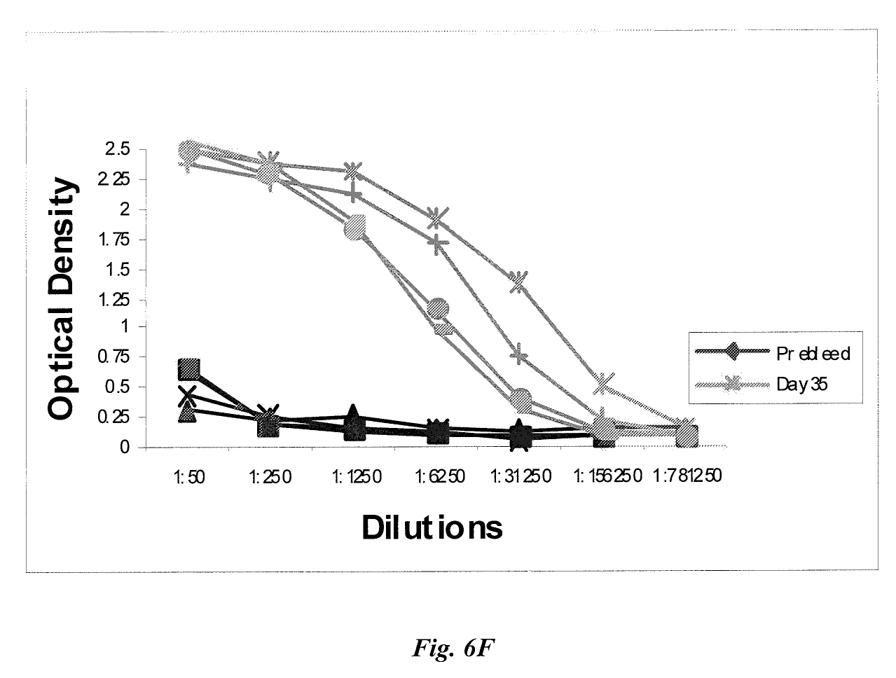

Guinea pigs were divided into two groups: Group 1, corresponding to FIGS. 6A-6C, received only Fluzone vaccine injections; Group 2, corresponding to FIGS. 6D-6E, received Fluzone vaccine injections and electrical stimulation via saline-containing devices.

Pre-treatment serum samples were taken from each group for use as controls when measuring antibody titers throughout the study (termed ‘pre-bleed’). Guinea pigs from both group were injected intramuscularly with suboptimal 0.1 μg doses of Fluzone vaccine (0.033 μg of each strain) as a prime.

Following injection, Group 2 received electrical stimulation at the site of injection via a saline-containing iontophoretic patch, delivering a current of 1 mA (density 0.3 mA/cm2) for 30 minutes (average recorded voltage of ˜4 volts).

Both groups of animals received a Fluzone vaccine boost intradermally at a time designated as time=0 days. Serum samples were taken from animals in each group for measurement of influenza antibody titer. Titers, indicated as optical density measurements, are shown for Groups 1 and 2 in FIGS. 6A and 6D, respectively.

Immediately after sampling for antibody titer measurements, the animals in Group 2 received electrical stimulation at the site of injection via a saline-containing iontophoretic patch, delivering a current of 1 mA (density 0.3 mA/cm2) for 30 minutes (average recorded voltage of ˜4 volts).

Further serum samples were taken from animals in each group at 21 days and 35 days following the Fluzone vaccine boost at day 0. Antibody titers for animals in Groups 1 and 2 at 21 days are shown in FIGS. 6B and 6E, respectively. Antibody titers for animals in Groups 1 and 2 at 35 days are shown in FIGS. 6C and 6F, respectively.

For measurement of antibody titer at days 0, 21, and 35, 500 μL of blood was drawn from each animal. Each sample was centrifuged, yielding ˜250 μL of serum for analysis by ELISA.

What is claimed is:

1. A method for enhancing an immune response of a mammal to a vaccine or immunogen comprising:
administering the vaccine or immunogen through a biological interface of the mammal;
positioning an electrode device on the surface of the biological interface at or near the site of administration; and
activating the electrode device to provide an electrical potential to the biological interface;
wherein the electrode device comprises a first electrode assembly comprising a first electrode element and a second electrode assembly comprising a second electrode element; and
wherein activating the electrode device to provide an electrical potential to the biological interface enhances the immune response of the mammal to the vaccine or immunogen.

2. The method of claim 1, further comprising: administering more than one dose of a vaccine or immunogen.

3. The method of claim 1, further comprising: administering more than one vaccine or immunogen.

4. The method of claim 3 wherein the vaccines or immunogens are administered concurrently.

5. The method of claim 3 wherein the vaccines or immunogens are administered consecutively.

6. The method of claim 1, further comprising: administering an adjuvant through the biological interface of the mammal at or near the site of administration of the vaccine or immunogen.

7. The method of claim 6 wherein the adjuvant is administered prior to administration of the vaccine or immunogen.

8. The method of claim 6 wherein the adjuvant is administered concurrent with administration of the vaccine or immunogen.

9. The method of claim 6 wherein the adjuvant is administered following administration of the vaccine or immunogen.

10. The method of claim 1 or 6 wherein administration is by injection.

11. The method of claim 1 or 6 wherein administration is by iontophoresis.

12. The method of claim 1 wherein the biological interface is a skin.

13. The method of claim 1 wherein the biological interface is a mucosal membrane.

14. The method of claim 1 wherein the electrical potential is applied at a constant level.

15. The method of claim 1 wherein the electrical potential is applied in pulses.

16. The method of claim 1 wherein the electrode device further comprises a first electrolyte reservoir comprising a first electrolyte composition, the first electrolyte reservoir positioned between the first electrode element and a surface of the first electrode assembly to be affixed to the biological interface.

17. The method of claim 16 wherein the electrode device further comprises a second electrolyte reservoir comprising a second electrolyte composition, the second electrolyte reservoir positioned between the second electrode element and a surface of the second electrode assembly to be affixed to the biological interface.

18. The method of claim 16 or 17 wherein the first electrolyte composition and/or the second electrolyte composition comprise a physiological ion.

19. The method of claim 18 wherein the physiological ion is selected from sodium, chloride, or phosphate.

20. The method of claim 16 or 17 wherein the first electrolyte composition comprises fumarate and polyacrylate ions and wherein the second electrolyte and wherein the second electrolyte composition comprises ascorbate and polyacrylate ions.

21. The method of any one of claims 1, 16, and 17 wherein the electrode device further comprises a means for affixing the electrode device to the biological interface of the mammal.

22. The method of claim 21 wherein the means for affixing the electrode device to the biological interface of the mammal comprises an adhesive.

23. The electrode device of any one of claims 1, 16, and 17 wherein the electrode device is in the form of a patch.

24. An electrode device for use in a method for enhancing an immune response of a mammal to a vaccine or an immunogen, the electrode device comprising:
a first electrode assembly comprising a first electrode element; and
a second electrode assembly comprising a second electrode element.

25. The electrode device of claim 24 for use in a method for enhancing an immune response of a mammal to a vaccine or an immunogen, the method comprising:
administering the vaccine or immunogen through a biological interface of the mammal;
positioning the electrode device to the surface of the biological interface at or near the site of administration; and
activating the electrode device to provide an electrical potential to the biological interface.
